(19) United States
(12) Patent Application Publication
(10) Pub. No.: US 2003/0108586 A1
(43) Pub. Date: Jun. 12, 2003

(54) Method of long-term reversible contraception for animals

(76) Inventor: Jacques Ramey, Omaha, NE (US)

Correspondence Address: Mark Frederiksen, PO Box 938, Council Bluffs, IA, 51502-0938, US

(21) Appl. No.: 10/014,694

(22) Filed: Dec. 11, 2001

(51) Int. Cl. A61K 38/24

(52) U.S. Cl. 424/423; 514/12

(57) ABSTRACT

A method of preventing fertility in a non-human animal includes administering a GnRH antagonist to the animal in an amount sufficient to prevent fertility.

### CROSS-REFERENCES TO RELATED APPLICATIONS

(Not applicable)

### STATEMENT AS TO RIGHTS TO INVENTIONS MADE UNDER FEDERALLY SPONSORED RESEARCH AND DEVELOPMENT

(Not applicable)

### BACKGROUND OF THE INVENTION

(1) Field of the Invention

The present invention relates generally to methods for preventing conception in animals, and more particularly to an improved method for contraception that is long-term and reversible.

(2) Background Information

The standard method for controlling animal fertility currently used in the prior art is by surgical removal of the animal's reproductive organs. If pet owners want a safe and efficacious method of temporarily preventing reproductive capabilities, few options are currently available. Nonsurgical means of controlling reproduction that are currently available include the use of intravaginal devices and a few pharmacological agents (generally composed of steroids).

Only two drugs are approved in the U.S.A. as a means of controlling small animal reproduction: (1) Ovaban™ (megestrol acetate; a product produced by Schering-Plough Animal, Union, N.J.), a synthetic progestin and Mibolerone™ (Cheque drops; a product produced by Pharmacia and Updon, Peapack, N.J.), a synthetic androgen (both of which are approved for reproductive suppression in female canines). These drugs are administered daily, usually by mixing them in the dog's food. The dosage is based on weight and the length of administration depends on when the drug is given in relation to the animal's reproductive cycle. An 8-32 day course of Ovaban™ is required to prevent fertility. This drug should not be administered for more than two consecutive treatment cycles. In contrast, Mibolerone™ is approved for daily administration for up to 2 years and must be instituted for at least 30 days to prevent pregnancy. This drug is not recommended for use in dogs that are to be used for future breeding.

The administration of these synthetic steroids has been noted to have several serious adverse effects. Mibolerone™ has been reported to result in clitoral hypertrophy in bitches. The use of synthetic progestins like Ovabant™ can also result in the development of mammary tumors as well as endometrial hyperplasia.

These two oral forms of contraception have only been recommended for use in female dogs and have not been recommended in male dogs. In fact, there are no nonsurgical means of contraception approved in this country for male dogs or male animals in general.

### BRIEF SUMMARY OF THE INVENTION

It is therefore a general object of the present invention to provide an improved pharmacological method of controlling reproduction that can be used in both male and female animals.

Yet another object is to provide a method of contraception in animals using a gonadotropin releasing hormone (GnRH) antagonist.

A further object of the present invention is to provide a method of controlling reproduction in animals that is long-term and reversible.

These and other objects of the present invention will be apparent to those skilled in the art.

The method of preventing fertility in a non-human animal of the present invention includes the step of administering a GnRH antagonist to the animal in an amount sufficient to prevent fertility for the desired period of time. Typically, this time period is a minimum of 6-12 months. In the preferred embodiment of the invention, the animal is a female domestic pet, and the drug is introduced by one of three delivery systems: (1) a long-term injectable drug, (2) a dissolvable capsule, or (3) a removable, non-dissolvable capsule.

### BRIEF DESCRIPTION OF THE SEVERAL VIEWS OF THE DRAWING

(Not applicable)

### DETAILED DESCRIPTION OF THE INVENTION

GnRH is a hypothalamic decapeptide hormone (a 10 amino acid molecule), which regulates the synthesis and release of gonadotropins from the pituitary gland, follicle stimulating hormone (FSH) and leutinizing hormone (LH). Gonadotropins are hormones that, in turn, control gonadal (ovarian and testicular) function. Normally, GnRH is released from the brain in a pulsatile fashion to effect normal synthesis and secretion of the gonadotropins. When native GnRH is administered in a continuous drug-like fashion, there is GnRH receptor complex internalization (loss) resulting in “down-regulation” of pituitary GnRH. The pituitary becomes subsequently desensitized (unresponsive), leading to decreased synthesis and secretion of gonadotropins. Clinically, the result is the development of iatrogenic (drug induced) hypogonadotropic hypogonadism; i.e., reversible sterility.

Gonadotropin-releasing hormone agonists and antagonists have been developed for manipulation of gonadal function in the treatment of various reproductive system disorders in humans, including precocious puberty, prostate cancer, endometriosis, hirsutism, and infertility. GnRH agonists have been available for clinical use for the past fifteen years, while GnRH antagonists have only just recently been introduced for human clinical use.

GnRH agonists are derived from native GnRH with substitutions of amino acids at positions 6 and/or 10 of the decapeptide chain. These substitutions result in an extended agonist half-life as compared to the half-life of native GnRH. Due to the extended half-life, administration of these agonists simulates the down-regulatory action of continuously administered native GnRH. GnRH agonists facilitate treatments requiring suppression of gonadotropins or gonadal steroid secretion.

An inherent disadvantage of GnRH agonists in humans is an initial stimulatory or “flare” effect that occurs before “down regulation,” resulting in an increased release of the gonadotropins. In non-human species this initial stimulatory effect, however, is sometimes used to enhance the reproductive cycle of female animals to induce ovulation. The “flare” affect generally subsides within 7-10 days and is then followed by down-regulation. This 10-day latency period to achievement of pituitary suppression can be inconvenient and delay therapeutic effects in humans. It also would not make GnRH agonists a good first choice as contraception in animals because of the initial fertility-enhancing “flare” affect. Furthermore, the reversal of pituitary suppression after discontinuation of the agonist is protracted for up to 14 days if fertility is desired in females and possibly longer for males.

Unlike native GnRH and GnRH agonists, GnRH antagonists appear to have no intrinsic physiologic action other than that of occupying the pituitary GnRH receptor sites, and are thought to block the action of native GnRH by classical competitive blockade. They induce a rapid, reversible suppression of gonadotropin secretion within 12-24 hours, providing several advantages over GnRH agonists. Upon discontinuation of the antagonist, pituitary LH and FSH levels are fully recovered in 48 hours.

Two GnRH antagonists are currently available in the U.S.A. One is ganirelix acetate (manufactured under the brand name Antagon™ by Organon, Inc. of West Orange, N.J.) and the other is cetrorelix acetate (manufactured under the brand name Cetrotide™ by Serono, Inc. of Norwell, Mass.). Antagon™ is derived from native GnRH with substitutions of amino acids at positions 1, 2, 3, 6, 8 and 10. Cetrotide™ has substitutions of amino acids at positions 1, 2, 3, 6, and 10. Both are currently FDA approved for inhibition of the LH surges in women undergoing fertility treatment using controlled ovarian hyperstimulation.

Future indications of GnRH antagonists in humans should expand. The antagonist will likely prove useful for the treatment of other disorders requiring gonadotropin or gonadal steroid suppression. The rapid onset and reversibility of action promises greater versatility for therapeutic manipulation of gonadal function. However, like GnRH agonists, the GnRH antagonists do not make useful forms of contraceptives in humans because of their total suppression of reproductive function (i.e., medical castration). Humans would not tolerate the subsequent side effects that would result from this type of hormonal suppression.

While the GnRH antagonists are not useful as a contraceptive in humans, the inventor herein has discovered that the same hormone would make an excellent choice as a new drug for a contraceptive in non-human animals. The advantages of GnRH antagonists as a contraceptive far outweigh other forms of non-surgical animal contraceptives. First, the use of a GnRH antagonist is safe with a rapid onset of its contraceptive action. Second, it can be used in both males and females, with females benefiting sooner than males. The side effects while on the drug are no different than those encountered by animals that are spayed or neutered. After discontinuation of the drug, reproductive function resumes within 24-48 hours; although the actual time in which an animal becomes fertile depends on the length of the ovulatory cycle in females, and in males, the functional time to resume spermatogenesis.

Another great advantage of the use of GnRH antagonists as a contraceptive in animals is that the molecular structure of native GnRH is highly conserved between species. Therefore, the same GnRH antagonist could be used to control reproductive function in many different species as well as disrupt reproductive function in both male and female individuals within a species.

Several different routes of delivery for a contraceptive using a GnRH antagonist could be employed. The three preferred routes include: (1) the use of a long-term injectable form of the drug, (2) an implantable dissolvable capsule, and/or (3) an implantable removable (non-dissolvable) capsule. All three delivery systems are well known in the art and are currently utilized for different types of drugs now used in both animals and humans. Preferably, the length of time that the drug would function would be at least 6 to 12 months.

An injectable form of the GnRH antagonist is similar to what is currently used in humans employing GnRH agonists such as depot leuprolide acetate (manufactured under the brand name Lupron™ by TAP of Deerfield, Ill.) and/or a synthetic progestin depot medroxprogesterone (manufactured under the brand name Provera™ by Pharmacia and Upjohn of Kalamazoo, Mich.). The drug is suspended in oil (peanut or linseed), and a fixed dosage is administered via intramuscular injection. In humans, the most common dose used for depot Lupron™ is 3.75 mg monthly, which results in complete suppression of ovarian function for a period of at least one month in a 60-80 kg woman. A similar route of delivery is used for a GnRH antagonist in a non-human animal, but the exact dosage is determined by the practitioner based on the length of action desired, and the size, weight and type of animal on which the drug is used. This dosage is easily determined for non-human animals by the ordinary practitioner, based upon the results achieved in humans.

A second route of delivery is a dissolvable implantable capsule. A GnRH antagonist is impregnated into an inert matrix and inserted under the skin surgically. In humans, a similar type of delivery system is marketed in the U.S.A. for ovarian suppression using a GnRH agonist. Goserelin acetate 3.6 mg (manufactured under the brand name Zoladex™ by Zeneca Pharmaceuticals of Wilmington, Del.) is designed for subcutaneous insertion with continuous release of the drug over a 28-30 day period of time. The capsule is placed surgically under the skin monthly. A disadvantage of this type of delivery system for the contraceptive (as well injectable contraceptives, i.e., depot Provera™) is that once the drug is administered, the length of action is predetermined and cannot be discontinued before the end of the scheduled length of action.

A third route of delivery commonly available is the nondissolvable implant. The significant advantage of this form of delivery system is that it is possible to reinstate reproductive capabilities sooner than the predetermined contraceptive action, by removing the capsule. Once the capsule is removed, the animal's reproductive function would then return within a short period of time. In humans, GnRH agonists are not delivered in this fashion; however, a contraceptive containing a synthetic progestin has been developed, i.e., Norplant™ (levonogesteril; manufactured by Wyeth-Ayerst, Philadelphia, Pa.). In this form of human contraceptive, the progestin is contained within a silastic implant that is surgically implanted under the skin. The steroid diffuses out of the capsule producing a contraceptive action in humans for a year or more. If contraception is no longer desired before the intended length of time, the implant can be removed and fertility will return shortly after removal. The same results are expected when used with a GnRH antagonist as a contraceptive in animals, using a nondissolvable implant.

Alza Corporation (Mt. View, Calif.) has patented a sustained delivery system using a nondissolvable implantable capsule (see U.S. Pat. No. 5,985,305). This delivery system could easily be utilized in the method of the present invention. The capsule could be implanted under the skin of male and female test animals (dogs/cats) using a small trochar similar to that used in the past in humans for the Norplant™ system or Zoladex™. By using a prolonged in vivo delivery of a GnRH antagonist, a very effective long term and/or short-term form of contraceptive should result, that would be safe and easily reversible.

As discussed above, these are three preferred routes of delivery for a GnRH antagonist used as a contraceptive in non-human animals. Others could be considered as well.

Whereas the invention has been shown and described in connection with the preferred embodiments thereof, many modifications, substitutions and additions may be made which are within the intended broad scope of the appended claims.

I claim:

1. A reversible method for preventing fertility in a non-human animal, comprising administering a gonadotropin releasing hormone antagonist to the animal in an amount sufficient for preventing fertility.

2. The method of preventing fertility of claim 1, wherein the animal is a domestic pet.

3. The method of preventing fertility of claim 2, wherein the animal is a female and the amount of the hormone is sufficient to prevent pregnancy.

4. The method of preventing fertility of claim 3, wherein the amount of antagonist administered is sufficient to prevent pregnancy for at least 6 months.

5. The method of preventing fertility of claim 4, wherein the amount of antagonist administered is sufficient to prevent pregnancy for at least 12 months.

6. The method of preventing fertility of claim 5, wherein the step of administering the hormone includes placing an effective amount of the hormone in an implantible capsule and implanting the capsule in the animal.

7. The method of preventing fertility of claim 6, wherein the step of implanting the capsule includes the step of inserting the capsule subcutaneously in the animal.

8. The method of preventing fertility of claim 7, wherein the capsule is dissolvable, and wherein the hormone is impregnated into the dissolvable capsule.

9. The method of preventing fertility of claim 7, wherein the capsule is formed of a non-dissolvable material that permits perfusion of the hormone.

10. The method of preventing fertility of claim 9, wherein the capsule contains an oil-based solution, and wherein the hormone is injected into the solution within the capsule.

11. The method of preventing fertility of claim 5, wherein the step of administering the hormone includes suspending the hormone in oil and administering a fixed dosage via intramuscular injection.

12. The method of preventing fertility of claim 1, wherein the animal is a female and the amount of antagonist administered is sufficient to prevent pregnancy for at least 12 months.

13. The method of preventing fertility of claim 12, wherein the step of administering the hormone includes placing an effective amount of the hormone in an implantible capsule and implanting the capsule in the animal.

14. The method of preventing fertility of claim 13, wherein the step of implanting the capsule includes the step of inserting the capsule subcutaneously in the animal.

15. The method of preventing fertility of claim 13, wherein the capsule is dissolvable and impregnated with the hormone.

16. The method of preventing fertility of claim 13, wherein the capsule is formed of a non-dissolvable material that permits perfusion of the hormone.

17. The method of preventing fertility of claim 16, wherein the capsule contains an oil-based solution, and wherein the hormone is injected into the solution within the capsule.

18. The method of preventing fertility of claim 12, wherein the step of administering the hormone includes suspending the hormone in oil and administering a fixed dosage via intramuscular injection.
